(19) United States
(12) Patent Application Publication
(10) Pub. No.: US 2024/0173241 A1
(43) Pub. Date: May 30, 2024

(54) Multistable Photochromic Pigments For Intradermal Use

(71) Applicant: THE REGENTS OF THE UNIVERSITY OF COLORADO, a body corporate, Denver, CO (US)

(72) Inventors: Carson J. Bruns, Boulder, CO (US); Jesse Butterfield, Boulder, CO (US)

(21) Appl. No.: 18/552,500

(22) PCT Filed: Mar. 31, 2022
(86) PCT No.: PCT/US22/22802

§ 371 Date: Sep. 26, 2023

Related U.S. Application Data
(60) Provisional application No. 63/169,066, filed on Mar. 31, 2021.

(51) Int. Cl. A61K 8/70; A61K 8/49; A61K 8/81; A61K 8/891; A61M 37/00; A61Q 1/02

(52) U.S. Cl. CPC A61K 8/70; A61K 8/4926; A61K 8/8152; A61K 8/891; A61M 37/0015; A61M 37/0076; A61Q 1/02; A61K 2800/438; A61K 2800/91; A61M 2037/0007; A61M 2037/0061

(57) ABSTRACT

Biocompatible, bistable or multistable (P-Type) photochromic nanoparticles or microparticles that can be embedded in the skin using techniques such as those used to create a tattoo with tattoo ink. The color of the “tattoo” using the biocompatible bistable or multistable photochromic microparticles can be written, erased or changed, and re-written locally in the skin by irradiation with light of appropriate wavelengths. Some formulations with much higher sensitivity to UV light than visible light can also be used as short-term intradermal colorimetric UV dosimeters that can be reset and re-used on an hourly or daily basis. These particles can be uniform aggregates or polymers containing P-type photochromic dyes, microencapsulated or core-shell structures with cores containing P-type photochromic dyes, or solid particulate materials with P-type photochromic dyes adsorbed on the surface or within their pores.

### FIELD OF INVENTION

This invention relates generally to tattoo ink and medical and cosmetic applications thereof. More particularly, this invention relates to compositions and methods for producing biocompatible, photochemically bistable or multistable nano- and microparticles and inks derived thereof for intradermal use as optically re-writable tattoos and permanent makeup, biopsy markers, and intradermal colorimetric ultraviolet (UV) detectors and dosimeters.

### BACKGROUND OF THE INVENTION

Tattoo and permanent makeup inks, which comprise nano- or microscale pigment granules (typically suspended in a water or alcohol-based fluid) leave permanent or semi-permanent visible body markings when injected at sufficient depth into the skin. These pigments change the color of the skin by modulating the frequencies of visible light that are absorbed and reflected in the dermis, where the pigments remain located long-term after the skin heals from the injection procedure. While most intradermal pigments are used for body art and permanent cosmetics, they also have biomedical applications in pre-surgical demarcation of anatomical biopsy sites, correction of pigmentary disorders, and medical aesthetics applications such, as reconstructive surgery and hair loss concealment.

### SUMMARY OF THE INVENTION

The present invention provides biocompatible UV-activated bistable or multistable photochromic microparticles that can be embedded in the skin using techniques such as those used to create a tattoo with tattoo ink. The “tattoo” using the biocompatible photochromic microparticles would provide skin with the ability to change color when exposed to specific wavelengths of light.

Depending on the constitution of dyes and pigments contained within these particles, the color changes can occur anywhere over the UV-visible-near-infrared range of wavelengths, allowing visible-to-invisible, invisible-to-visible, or visible-to-visible transformations that may be controlled and programmed with appropriate light sources. When the photochromism of these particles are several orders of magnitude more sensitive to UV light than visible light, they can be used for UV dosimetry. These particles can be uniform polymers with bonded or embedded P-type photochromic compounds, crystalline or amorphous molecular aggregates containing P-type photochromic compounds, polymer or inorganic particles coated with P-type photochromic compounds, core-shell (encapsulated) particles comprising crystalline solid, amorphous solid, gel, liquid, or solution cores containing P-type photochromic compounds and coated with solid polymer- or mineral-based shells, or mesoporous particles containing P-type photochromic compounds, where the P-type photochromic compounds may optionally be accompanied by other small-molecule compounds such as stabilizers and dyes as photo-filters.

An exemplary biocompatible UV-absorbing microparticle is poly(methyl methacrylate) (PMMA) in combination with a commercially-available. Some examples of materials that could be used as P-type photochromic dyes include include diarylethenes such as those taught in Japan Patent JP3882746B2 and in [Irie, M.; Fukaminato, T.; Matsuda, K.; Kobatake, S. Photochromism of Diarylethene Molecules and Crystals: Memories, Switches, and Actuators. Chem. Rev. 2014, 114. 12174-12277; Kobatake, S.; Takami, S.; Muto, H.; Ishikawa, T.; Irie, M. Rapid and reversible shape changes of molecular crystals on photoirradiation. Nature 2007, 446, 778-781; Irie, S.; Irie, M. Ultrahigh Sensitive Color Dosimeters Composed of Photochromic Diarylethenes and Fluorescent Metal Complexes. Chem. Lett. 2006, 35, 1434-1435; Kawamura, I.; Kawamoto, H.; Fujimoto, Y.; Masanori, K.; Asai, K. Isomerization behavior of diarylethene-type photochromic compounds under X-ray irradiation: application to dosimetry, Jpn. J. Appl. Phys. 2020, 59, 046004; Jin, Y.; Qamar, I.; Wessely, M.; Adhikari, A.; Bulovic, K.; Punpongsanon, P.; Mueller, S. Photo-Chromeleon: Re-Programmable Multi-Color Textures Using Photochromic Dyes. UIST '19 2019. 12 pp. New Orleans, LA. USA.] fulgides/fulgimides such as those described in

[Yokoyama, Y. Fulgides for Memories and Switches. Chem. Rev. 2000, 100, 1717-1739] naphthopyrans such as those described in [Frigoli, M.; Maurel, F.; Berthet, J.; Delbaere, S.; Marrot. J.; Oliveira, M. M. The control of photochromism of [3H]-naphthopyran derivatives with intramolecular CH-π bonds. Org. Lett. 2012, 14, 4150-4153; Frigoli, M.; Marrot, J.; Gentili, P. L.; Jacquemin, D.; Vagnini, M.; Pannacci, D.; Ortica, F. P-Type Photochromism of New Helical Naphthopyrans: Synthesis and Photochemical, Photophysical and Theoretical Study. ChemPhysChem 2015, 16, 2447-2458] and hydrazones such as those described in [van Dijken, D. J.; Kovaříček, P.; Ihrig. S. P.; Hecht. S. Acylhydrazones as Widely Tunable Photoswitches. J. Am. Chem. Soc. 2015, 137, 14982-14991; Qian, H.; Pramanik, S.; Aprahamian, I. Photochromic Hydrazone Switches with Extremely Long Thermal Half-Lives. J. Am. Chem. Soc. 2017, 139. 9140-9143; Shao, B.; Qian, H.; Li, Q.; Aprahamian, I. Structure Property Analysis of the Solution and Solid-State Properties of Bistable Photochromic Hydrazones. J. Am. Chem. Soc. 2019, 141. 8364-8371] Other suitable polymer encapsulant materials include polyacrylates and polyacrylamides, poly(dimethyl siloxane) (PDMS) and similar silicone rubbers, melamine-formaldehyde and other amino resins, epoxy resins, cross-linked polyethylene glycol (PEG) networks and related biocompatible networks, as well as poly(lactic acid) (PLA), poly(lactic-co-glycolic acid) (PLGA), methacrylamide chitosan, and many others.

In a first aspect the present invention provides a bistable photochromic particle comprising poly(methyl methacrylate) (PMMA) in combination with a P-type photochromic dye. The P-type photochromic dye can be dyes such as diarylethenes, fulgides, fulgimides, naphthopyrans, hydrazones, and combinations thereof.

In a second aspect the present invention provides a bistable photochromic particle comprising a polymer in combination with a P-type photochromic dye consisting of a diarylethene compound. In an advantageous embodiment, the polymer used in the composition of the second aspect can be PDMS and other silicone rubbers, Melamine-formaldehyde and other amino resins, cross-linked PEG and other biocompatible networks, PLA, PLGA, Methacrylamide chitosan, epoxy resins, PAA, PMMA, and other acrylate-based and acrylamide-based polymers and networks, and combinations thereof.

In a third aspect the present invention provides a bistable photochromic particle composition comprising a polymer and P-type photochromic dye in combination with stable UV- or Visible-absorptive material(s), wherein the UV- or Visible-absorptive material(s) acts as a filter(s) to tune spectral sensitivity or color appearance of the composition. The UV-absorptive material can be hydroxybenzophenone, hydroxyphenyl-s-triazine, 2-(2-hydroxyphenyl)benzotriazole, oxalanilide, Aminobenzoic acid, Avobenzone, Cinoxate, Dioxybenzone, Homosalate, Meradimate, Octocrylene, Octinoxate, Octisalate, Oxybenzone, Padimate O, Ensulizole, Sulisobenzone, Cerium Dioxide, Titanium dioxide, Trolamine salicylate, Zinc oxide, layered double hydroxides, derivatives of the aforementioned compounds and combinations thereof. The visible-absorptive materials can be azo dyes, perylenes, anthraquinones, cyanines, triarylmethines, commercial pigments, Pigment Red, Pigment Orange, Pigment Yellow, Pigment Blue, Pigment Green, Pigment Violet, Pigment Black, Pigment White, and combinations thereof. In an advantageous embodiment the bistable photochromic particle composition according to the third aspect includes a photo-stabilizer to inhibit photodegradation of the polymer, thereby increasing the service life of the particle. The photostabilizer can be a hindered amine. In an advantageous embodiment the hindered amine is 2,2,6,6-tetramethylpiperidine, a derivative of 2,2,6,6-tetramethylpiperidine, or an alkylated or hydroxylamine analog of 2,2,6,6-tetramethylpiperidine.

### BRIEF DESCRIPTION OF THE DRAWINGS

For a fuller understanding of the invention, reference should be made to the following detailed description, taken in connection with the accompanying drawings, in which:

FIG. 1 is an illustration providing a graphical representation of different P-type photochromic microparticle formulations. (A) Polymer nano- or microparticles, (B) Molecular aggregates (crystalline or amorphous), (C) Surface-coated nano- or microparticles (D) Core-shell nano- or microparticles, (E) Mesoporous nano- or microparticles.

FIG. 2 is a graph (A) and an image (B) showing characterization data of P-type photochromic PMMA nanoparticles prepared according to the example procedure. (A) Size distribution data for PMMA particles prepared according to the example procedure. (B) Scanning electron micrograph of the particles obtained from dried aqueous ink dispersions of the nano- and microparticles of Formulation A. All particles are made of PMMA and contain diarylethene compound DAE-0001 (Yamada Chemical, 10 wt %) as the P-type photochromic dye.

FIG. 3 is a set of images (A and B) and graphs (C and D) showing P-type photochromic nanoparticle tattoo inks prepared according to the examples presented below. (A) Photograph of a vial of P-type photochromic PMMA nanoparticle tattoo ink prepared according to the example procedure (Formulation A) prior to activation by UV light, which appears cloudy white due to scattering. (B) Photograph of the same tattoo ink after UV activation, which appears blue (dark gray in gray-scale) due to the thermally irreversible photochemical reaction of the P-type photochromic dye embedded in the polymer microparticles. The inks are formulated at a particle concentration of approximately 10 wt % and contain the biocompatible surfactant PVA (0.1 wt%) as stabilizer. (C) Normalized UV-Vis absorption spectrum of a dilute suspension of the P-type photochromic PMMA nanoparticles in deactivated/colorless (black line) and activated/colored (dashed line) states. (D) Steady-state shear rheological frequency sweep reveals the shear thinning behavior of the ink.

FIG. 4 is a set of four images showing photographs of an ex vivo porcine skin sample tattooed with a bistable P-type photochromic PDMS microparticle tattoo ink (as in FIG. 3) prepared according to the example procedure described below. (A) Photograph of a square-shaped tattoo of the bistable tattoo ink. The tattoo is minimally visible to the naked eye (pale white due to scattering; this effect will be minimized once the healing process is complete in vivo). (B)

Photograph of the same tattoo after UV activation through a transparency mask in the shape of an eight-pointed star. Exposed areas become a stable blue color due to the photochromic properties of the intradermally implanted microparticles. (C) Photograph of the same tattoo after deactivation with red light. The tattoo returns to the colorless state. (D) Photograph of the same tattoo after re-activation with UV light in the shape of a five-pointed star, showing that the writing, erasing, and re-writing process is photochemically reversible.

FIG. 5 is a set of six graphs (A-F) comparing measured and modeled data for the UV activation of P-type photochromic dyes with and without UV and color filters. The UV-activated color change is quantified by ΔE*_{ab} in CIELAB chromaticity space for measured data, while it is expressed in terms of the relative concentration. [C], of the photoactivated species in the modeled data. (A) ΔE*_{ab} vs. time for a P-type photochromic dye (1,2-Bis(2-methyl-5-phenyl-3-thienyl)-perfluorocyclopentenediarylethene, DAE-0001) embedded in a PMMA matrix in natural sunlight in Boulder, Colorado. The time to 85% complete activation, 185, is 67 s. (B) ΔE*_{ab} vs. time for DAE-0001 with 50% UV filter (185 =142 s). (C) Simulated activation data ([C] vs. time) for DAE-0001 based on empirical absorptivity and quantum yield data using the photochemical kinetics equation specified in Example 4 at a fixed solar irradiance (t₈₅=67 s). (D) Simulated activation data ([C] vs. time) for DAE-0001 with a reduced UV irradiance attributable to a simulated UV filter (t₈₅=67 s). (E) Simulated activation data ([C] vs. time) for a P-type photochromic dye with a low cycloreversion quantum yield, based on empirical absorptivity and quantum yield data for (1,2-Bis(2-methoxy-5-phenyl-3-thienyl)-perfluorocyclopentenediarylethene using the photochemical kinetics equation specified in Example 4 at a fixed solar irradiance (t₈₅=217 s). (F) Simulated activation data ([C] vs. time) for the same low-cycloreversion dye with a 33% reduced UV irradiance and 50% reduced visible irradiance attributable to a simulated UV and color filter combination (t₈₅=1092 s).

### DETAILED DESCRIPTION OF THE PREFERRED EMBODIMENT

Tattoos are formed using intradermal nanoparticles (typically 20 nm-900 nm in diameter) in the form of color additives, most often borrowed from the pigment manufacturing industry. Tattoo pigments are typically inserted in the dermis by repeatedly puncturing the skin with a needle or array of needles carrying a tattoo ink comprising a dispersion of these pigments, although alternative needle-free injection strategies are in development. Without intervention, tattoos leave permanent markings on the skin because the pigments undergo repeated cycles of capture and release by dermal melanophages with minimal migration in the dermis. Long-term tattoo fading is caused by clearance of the pigments via drainage into the lymph nodes by these immune cells, and this process may be accelerated by pigment photodegradation associated with laser tattoo removal treatment as well as UV exposure in sunlight.

Tattoos, permanent makeup, and related biomedical applications typically rely on conventional industrial pigment colorants, although some pre-biopsy tattoo pigments have been engineered to exhibit fluorescence. Most tattoo and permanent makeup pigments are stable colorants that do not readily undergo color-changing chemical or photochemical reactions in the dermis.

“T-type” photochromic dyes undergo a photochemical reaction that generates a color change when activated by light of an appropriate wavelength. In T-type photochromic dyes, this photochemical reaction is thermally reversible, so the dye returns to its original state spontaneously when the activating light is removed.

P-type photochromic dyes are not thermally reversible, and therefore do not spontaneously return to their original color after activation. Instead, reversal of photochemical activation in P-type dyes is accomplished by a second photochemical activation of a different wavelength range. Owing to their thermal irreversibility, P-type dyes are bistable in appropriate lighting conditions, and are therefore suitable for dosimetry. [Japan Patent JP3882746B2; Irie, S.; Irie, M. Ultrahigh Sensitive Color Dosimeters Composed of Photochromic Diarylethenes and Fluorescent Metal Complexes. Chem. Lett. 2006, 35, 1434-1435; Kawamura, I .; Kawamoto, H .; Fujimoto, Y .; Masanori, K .; Asai, K. Isomerization behavior of diarylethene-type photochromic compounds under X-ray irradiation: application to dosimetry, Jpn. J. Appl. Phys. 2020, 59, 046004].

The present invention provides a photochemically patternable and re-writable pigment that can be used in the skin. In a first aspect the technology utilizes formulations of P-type photochromic nanoparticles and/or microparticles (See Example 1, below). In further aspects the present invention provides inks (See Example 2, below) utilizing dispersions of these particles, such as in the first aspect, that enable implantation in the dermis. In still further aspects the present invention provides techniques for implanting the inks in the dermis, including conventional tattooing, permanent make-up, threading, and microneedle patches (See Example 3, below). In the final aspect, the present invention provides methods of utilizing the wavelength sensitivity of bistable or multistable photochromic tattoos to write, erase, and re-write particular colors and patterns in the skin for body art, cosmetic, or biopsy site marking purposes, or to record intradermal UV dosimetry information in a suitable embodiment of the photochromic tattoo (See Example 4, below).

### EXAMPLE 1—MATERIALS AND METHODS

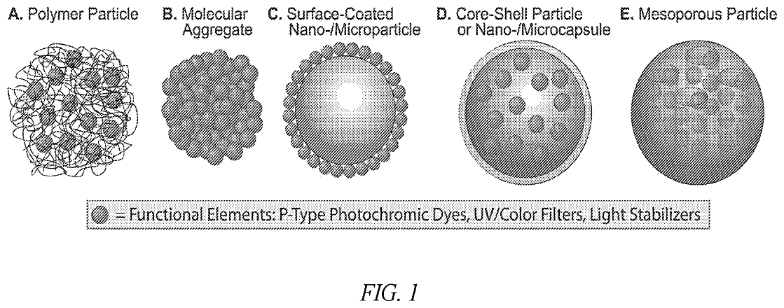

The present invention provides formulations for P-type photochromic nano- or microparticles (see e.g., FIG. 1) for intradermal use. The mean particle diameters will advantageously fall within the range of approximately 20 nm to 10 microns in order to (i) facilitate implantation in the dermis by tattooing or other means and (ii) to remain located semi-permanently or permanently in the dermis. As particle size gets below this size scale the particles are more easily cleared by the immune system. On the other hand, larger particles (e.g. in excess of about 10 microns) may lead to excessive granuloma or keloid reactions. The particles can contain “functional elements”, depicted as darker spheres in FIG. 1. These functional elements comprise, minimally, a P-type photochromic dye. By “P-type photochromic dye” it is meant as any compound that meets the following two criteria: (i) the compound undergoes a photochemical reaction that changes its spectral absorbance profile when activated by a particular wavelength or range of wavelengths of light, and (ii) the photochemical reaction undergone by the compound is not thermally reversible but can be reversed by photochemical de-activation at a different wavelength or range of wavelengths than those used for activation. Classes of suitable P-type photochromic dyes that can be used as functional elements include diarylethenes [Irie, M.; Fukaminato, T.; Matsuda, K.; Kobatake, S. Photochromism of Diarylethene Molecules and Crystals: Memories, Switches, and Actuators. Chem. Rev. 2014, 114, 12174-12277], fulgides/fulgimides such as those described in [Yokoyama, Y. Fulgides for Memories and Switches. Chem. Rev. 2000, 100, 1717-1739.], naphthopyrans [Frigoli, M.; Maurel, F.; Berthet, J.; Delbaere, S.; Marrot, J.; Oliveira, M. M. The control of photochromism of [3H]-naphthopyran derivatives with intramolecular CH-π bonds. Org. Lett. 2012, 14, 4150-4153] and [Frigoli, M; Marrot, J.; Gentili, P. L.; Jacquemin, D.; Vagnini, M; Pannacci, D.; Ortica, F. P-Type Photochromism of New Helical Naphthopyrans: Synthesis and Photochemical, Photophysical and Theoretical Study. ChemPhysChem 2015, 16, 2447-2458], and hydrazones [van Dijken, D. J.; Kovaříček, P.; Ihrig. S. P.; Hecht, S. Acylhydrazones as Widely Tunable Photoswitches. J. Am. Chem. Soc. 2015, 137, 14982-14991; Qian, H .; Pramanik, S.; Aprahamian, I. Photochromic Hydrazone Switches with Extremely Long Thermal Half-Lives. J. Am. Chem. Soc. 2017, 139, 9140-9143; Shao, B.; Qian, H.; Li, Q.; Aprahamian, I. Structure Property Analysis of the Solution and Solid-State Properties of Bistable Photochromic Hydrazones. J. Am. Chem. Soc. 2019, 141, 8364-8371]. These P-type photochromic dyes may be used as the sole coloring elements to produce a single type of color change (bistable). In addition, multiple P-type photochromic dyes may be combined to further tune the wavelength sensitivity of the particles and access multistable coloring regimes through selective activation and/or deactivation of a subset of the dye component mixture, such as through the application of a multicomponent P-Type photochromic coating [Jin, Y.; Qamar, I.; Wessely, M.; Adhikari, A.; Bulovic, K.; Punpongsanon, P.; Mueller, S. Photo-Chromeleon: Re-Programmable Multi-Color Textures Using Photochromic Dyes. UIST '19 2019. 12 pp. New Orleans, LA, USA].

In addition to the P-type photochromic dyes, the formulations may also include any combination of the following functional elements:

UV Absorbers. UV absorbers may be included to tune the spectral distribution of the particles in the UV range, which can influence the kinetics and extent of photoactivation, as well as the stability of the particles to UV photodegradation. Various classes of UV absorbers are possible and appropriate for inclusion as UV-absorptive additives. Organic UV absorbers can include FDA-approved over-the-counter sunscreen drugs (see [US Food and Drug Administration. Sunscreen Drug Products for Over-the-Counter Human Use: Proposed Rule. Federal Register 2019, 84, 6204-6275]), industrial additives for coatings, such as benzophenones, benzotriazoles, and phenyltriazines [Keck, J.; Kramer, H. E. A.; Port, H.; Hirsch, T.; Fischer, P.; Rytz, G. Investigations on Polymeric and Monomeric Intramolecularly Hydrogen-Bridged UV Absorbers of the Benzotriazole and Triazine Class. J. Phys. Chem. 1996, 100, 14468-14475; Schaller, C.; Rogez, D.; Braig, A. Hydroxyphenyl-s-triazines: advanced multipurpose UV-absorbers for coatings. J. Coat. Technol. Res. 2007, 5, 25-31], or polymers incorporating these moieties within their repeating units [Kim, E; Cho, S. Y.; Yoo, M. J.; Ahn, K.-H. Vinyl group-containing diarylethene and polymer thereof having excellent optical properties. U.S. Pat. No. 6,787,621B2. Filed 19 Sep. 2002]. Inorganic/mineral UV absorbers can include TiO₂[ Allen, N. S.; Edge, M.; Ortega, A.; Liauw, C. M.; Stratton, J.; McIntyre, R. B. Behaviour of nanoparticle (ultrafine) titanium dioxide pigments and stabilisers on the photooxidative stability of water based acrylic and isocyanate based acrylic coatings. Polym. Degrad. Stabil. 2002, 78, 467-478], ZnO [Becheri, A.; Dürr, M.; Lo Nostro, P.; Baglioni, P. Synthesis and characterization of zinc oxide nanoparticles: application to textiles as UV-absorbers. J. Nanopart. Res. 2007, 10, 679-689], doped SiO₂ [He, Q.; Yin, S.; Sato, T. Synthesis and photochemical properties of zinc-aluminum layered double hydroxide/organic UV ray absorbing molecule/silica nanocomposites. J. Phys. Chem. Solids 2004, 65, 395-402], CeO₂ [Goubin, F., et al., Experimental and Theoretical Characterization of the Optical Properties of CeO₂, SrCeO₃, and Sr₂CeO₄ Containing Ce⁴⁺ (f0) Ions. Chem. Mater. 2004, 16, 662-669], which may be either crystalline, polycrystalline, or amorphous. UV absorbers can also include organic/inorganic combinations (for example, sec [Mahltig, B., et al., Optimized UV protecting coatings by combination of organic and inorganic UV absorbers. Thin Solid Films 2005, 485, 108-114]), including layered double hydroxides [Feng. Y.; Li, D.; Wang, Y.; Evans, D. G.; Duan, X. Synthesis and characterization of a UV absorbent-intercalated Zn-Al layered double hydroxide. Polym. Degrad. Stabil. 2006, 91, 789-794; Li, D.; Tuo, Z.; Evans, D. G.; Duan, X. Preparation of 5-benzotriazolyl-4-hydroxy-3-sec-butylbenzenesulfonate anion-intercalated layered double hydroxide and its photostabilizing effect on polypropylene. J. Solid State Chem. 2006, 179, 3114-3120; Cao, T.; Xu, K.; Chen, G.; Guo, C.-Y. Poly(ethylene terephthalate) nanocomposites with a strong UV-shielding function using UV-absorber intercalated layered double hydroxides. RSC Advances 2013, 3, 6282-6285].

Color Filters. Other dyes that absorb visible or near-infrared wavelengths of light may also be added to further tune the activation and/or deactivation sensitivity of the P-type photochromic dye. For example, in the case of UV-activated/visible-deactivated photochromic dyes, the presence of a color filter can lower the overall dose of visible light that is supplied to the photoactive dye in sunlight, prolonging the lifetime of the photoactivated state in outdoor environments. Examples of a suitable families of color dyes with tunable transmission wavelengths are the azo dyes [Ashida, T. Azo compounds, dyes containing them, and colored compositions. Japan Patent JP 2013043969A. 4 Mar. 4 2013. Sumitomo Chemical Co., Ltd., Japan; Do Kim, Y. et al., Synthesis, application and investigation of structure-thermal stability relationships of thermally stable water-soluble azo naphthalene dyes for LCD red color filters. Dyes and Pigments 2011, 89, 1-8], perylenes [Choi, J.; Sakong, C.; Choi, J.-.; Yoon, C.; Kim, J. P. Synthesis and characterization of some perylene dyes for dye-based LCD color filters. Dyes and Pigments 2011, 90, 82-88], anthraquinones [Park, J.; Park, Y.; Park, J. Synthesis and physical property measurement of new red pigment based on anthraquinone derivatives for color filter pigments. Mol. Cryst. Liq. Cryst. 2011, 551, 116-122], cyanines [Kwon, H.-S .; Yoo, J.-S.; Lee, H.-Y.; Choi, J.-H. Synthesis of Innovative Colorants Based on Cyanine Dye and Their FRET Efficiency to Reduce the Emission of Fluorescence for LCD Color Filter. Bull. Kor. Chem. Soc. 2015, 36, 2545-2548], triarylmethines [Kong, N. S. et al., Development of dimeric triarylmethine derivatives with improved thermal and photo stability for color filters. Dyes and Pigments 2017. 144, 242-248] and many others. See [Zollinger, H. Color Chemistry: Synthesis, Properties, and Applications of Organic Dyes and Pigments. (3^{rd} Ed.) Weinheim: Wiley-VCH, 2001] for more examples of appropriate dyes and pigments that can be used as color filters.

Photo-stabilizers. It is often beneficial to mix plastic materials, including polymeric particles such as those described in this invention, with photo-stabilizers that can inhibit photodegradation to increase their service life [see e.g., Muasher, M.; Sain, M. The efficacy of photostabilizers on the color change of wood filled plastic composites. Polym. Degrad. Stabil. 2006, 91, 1156-1165; Andrady, A. L.; Hamid, S. H.; Hu, X.; Torikai, A. Effects of increased solar ultraviolet radiation on materials. J. Photochem. Photobiol. B 1998, 46, 96-103]. Hindered amines, particularly those derived from 2,2,6,6-tetramethylpiperidine and its alkylated or hydroxylamine analogs, are an advantageous class of photostabilizer. These photostabilizers scavenge undesired radicals generated in organic materials under UVA and UVB irradiation and are subsequently regenerated (see e.g., the Denisov cycle, which is explained in [Hodgson, J. L.; Coote, M. L. Clarifying the mechanism of the Denisov cycle: How do hindered amine light stabilizers protect polymer coatings from photo-oxidative degradation? Macromolecules 2010, 43, 4573-4583]), imparting them with long-lasting light stabilizing function [Klemchuk, P. P.; Gande, M. E. Stabilization mechanisms of hindered amines. Polym. Degrad. Stabil. 1988, 22, 241-274].

Preferably, the particles will be pharmaceutically acceptable and exhibit little to no toxicity, immunogenicity, or teratogenicity. Particles will also exhibit high chemical, physical, and photo stability in aqueous media in the temperature range of 20-40 ° C., which is representative of intradermal conditions. Particles exhibiting these characteristics should maintain their long-term function and biocompatibility in the skin. The functional elements can also be insoluble, (or rendered insoluble by chemical or encapsulation strategies, vide infra) in aqueous media to prevent them from partitioning into the interstitial fluid. It is preferred to minimize the scattering, reflectance, and refraction of the particles in addition to their visible absorption, in order to minimize their visibility in skin in cases where one of the stable color states is meant to be invisible. As scattering is highest at particle diameters near 100-200 nm [Dawson, P. L.; Acton, J. C. Impact of proteins on food color. Proteins in Food Processing, Second Ed. 2018, Elsevier Ltd. pp. 599-638], a preferred particle size is on the size scale of visible light or higher (e.g., 400 nm and above). To minimize excessive reflection and refraction, which would cause the particles to appear white (Mie scattering), the refractive indices of the particles in the visible range can closely match that of the dermis (1.36-1.41, see [Ding. H.; Lu, J. Q; Wooden, W. A.; Kragel, P. J.; Hu, X.-H. Refractive indices of human skin tissues at eight wavelengths and estimated dispersion relations between 300 and 1600 nm. Physics in Medicine and Biology 2006, 51, 1479-1489]).

Formulation A. Polymer Particle. The functional elements may be integrated within polymer or co-polymer particles of appropriate size (˜20-10,000 nm) by a number of strategies, which may be broadly classified into dispersion approaches and polymerization approaches [Rao, J. P.; Geckeler, K. E. Polymer nanoparticles: Preparation techniques and size-control parameters. Prog. Polym. Sci. 2011, 36, 887-913]. Dispersion approaches involve converting pre-formed polymers into nano- or microparticles from a homogenous solution by solvent evaporation in a spray or emulsion, or by precipitation with solvent exchange, salt, dialysis, or supercritical fluids. Dissolving the functional elements in the polymer phase during these processes will incorporate them (non-covalently) into the polymer matrix of the resulting nano- or micro-particles. Polymerization approaches to polymer particle synthesis typically rely on emulsions, in which nano- or micro-droplets of pre-polymer resins (monomers), typically dispersed in aqueous solutions, are directly polymerized into particles upon initiation of the polymerization. In this case, the functional elements may be dissolved into the monomer phase of the emulsion to incorporate them into the polymer matrix upon polymerization.

In both dispersion and polymerization approaches, the functional elements may also be incorporated directly into the main chain, side chain, or cross-links of the polymer structure by including them as monomers during polymer synthesis. In most cases, the functional elements could be modified with reactive functional groups in order to be covalently bound to the polymer or co-polymer. For example, functionalizing a diarylethene-based P-type photochromic dye with one or more acrylic or vinyl functional groups would enable its polymerization or co-polymerization with other acrylic or vinyl monomers (as in PMMA and many silicone rubbers) by catalysis or radical polymerization [Kim, E.; Cho, S. Y.; Yoo, M. J.; Ahn, K.-H. Vinyl group-containing diarylethene and polymer thereof having excellent optical properties. U.S. Pat. No. 6,787,621B2. Filed 19 Sep. 2002]. Alternatively, the functional elements may be coupled to a pre-synthesized polymer [Finden, J.; Kunz, T. K.; Branda, N. R.; Wolf, M. O. Reversible and Amplified Fluorescence Quenching of a Photochromic Polythiophene. Adv. Mater. 2008, 20, 1998-2002]. These covalent-attachment methods of incorporating functional elements are more expensive than the admixture approaches, but they lower the risk of any functional elements leaching out of the particles.

Advantageous polymer matrices in this formulation include poly(methyl methacrylate) (PMMA) and other methacrylate compounds (e.g., poly(methyl methacrylate, poly(isopropyl methacrylate), poly(isobutyl methacrylate)). PMMA is a biocompatible polymer [Frazer, R. Q.; Byron, R. T.; Osborne, P. B.; West, K. P. PMMA: An Essential Material in Medicine and Dentistry. Journal of Long-Term Effects of Medical Implants 2005, 15, 629-639]. Another class of advantageous polymers are poly(dimethylsiloxane) (PDMS) and other silicone rubbers, which are also biocompatible [Rahimi, A.; Mashak, A. Review on rubbers in medicine: natural, silicone and polyurethane rubbers. Plastics, Rubber and Composites 2013, 42, 223-230]. These polymer matrices are particularly appropriate because (i) their biocompatibility is well-established, (ii) their refractive indices of less than 1.5 are close to that of the dermis, [Polymer Database. Refractive Index of Amorphous Polymers. Polymerdatabase.com] (iii) they exhibit high long-term stability and (iv) they are relatively convenient and inexpensive to produce.

Formulation B. Molecular Aggregate. Small-molecule or oligomer functional elements that are solid at biological temperatures may be employed directly as aggregated particles when they are sufficiently insoluble in aqueous media and of sufficient size for dermal implantation. Most conventional colored tattoo pigments (red, yellow, green, blue, etc.) are made of small molecules. Even though the molecules are too small individually to serve as a tattoo pigment, they aggregate into crystalline or amorphous nano- or microparticles. The molecules are not soluble in water, so they remain associated with these “molecular aggregate” particles, and the particles do not dissolve. Some functional elements could be employed to the extent that they behave the same way as these ordinary pigments. The processes of rendering poorly water-soluble compounds into small particulates are known as nanosizing [Kesisoglou, F.; Panmai, S.; Wu, Y. Nanosizing—Oral formulation development and biopharmaceutical evaluation. Adv. Drug Deliv. Rev. 2007, 59, 631-644], or micronizing [Rasenack, N.; Müller, B. W. Micron-Size Drug Particles: Common and Novel Micronization Techniques. Pharm. Dev. Technol. 2004, 9, 1-13]. Molecular aggregates can be prepared as nano- or microparticles by (i) precipitation from a solvent into a non-solvent (ideally water), [Rabinow, B. E. Nanosuspensions in drug delivery. Nat. Rev. Drug Discov. 2004. 3, 785-796], (ii) spray-drying processes [Vehring, R. Pharmaceutical Particle Engineering via Spray Drying. Pharm. Res. 2007, 25, 999-1022], (iii) supercritical fluid techniques [Martín, A.; Cocero, M. J. Micronization processes with supercritical fluids: Fundamentals and mechanisms. Adv. Drug Deliv. Rev. 2008, 60, 339-350] or (iv) milling [Merisko-Liversidge, E.; Liversidge, G. G.; Cooper, E. R. Nanosizing: a formulation approach for poorly-water-soluble compounds. Eur. J. Pharm. Sci. 2003, 18,113-120]. These methods can be used to generate nano- or microcrystalline P-type photochromic particles or amorphous P-type photochromic particles containing mixtures of UV absorbers, color filters, and/or stabilizers.

Advantageous functional elements in the form of P-type photochromic compounds in this case are the family of diarylethenes, since they reliably undergo their P-type photochromic reactions in the solid state [Kobatake, S.; Takami, S.; Muto, H.; Ishikawa, T.; Irie, M. Rapid and reversible shape changes of molecular crystals on photoirradiation. Nature 2007, 446, 778-781; Irie, S.; Irie, M. Ultrahigh Sensitive Color Dosimeters Composed of Photochromic Diarylethenes and Fluorescent Metal Complexes. Chem. Lett. 2006, 35, 1434-1435; Kawamura, I.; Kawamoto, H; Fujimoto, Y.; Masanori, K ; Asai, K. Isomerization behavior of diarylethene-type photochromic compounds under X-ray irradiation: application to dosimetry, Jpn. J. Appl. Phys. 2020, 59, 046004].

Formulation C. Surface-Coated Particle. A monolayer or multilayer of P-type photochromic compounds and other functional elements can be adsorbed to the surface of a nano- or microparticle by chemical or physical means. Covalent attachment of the functional elements to the particle affixes the functional element to the particle surface. For example, a surface-coated particle can employ silica particles as the substrate. Silica is an appropriate material because (i) it is already employed as a thixotropic agent in tattoo inks [Piccinini, P.; Pakalin, S.; Contor, L.; Bianchi, I.; Senaldi, C. Safety of tattoos and permanent make-up: Final report. European Commission Joint Research Centre Science for Policy Report 2016, 1-118], (ii) it can be biocompatible (see [Gerion, D.; Pinaud, F.; Williams, S. C; Parak, W. J.; Zanchet, D.; Weiss, S.; Alivisatos, A. P. Synthesis and properties of biocompatible water-soluble silica-coated CdSe/ZnS semiconductor quantum dots. J. Phys. Chem. B 2001, 105. 8861-8871]) and (iii) it is readily functionalized by silanization with a wide variety of alkoxysilanes and halosilanes [Voort, Der, P. V.; Vansant, E. F. Silylation of the Silica Surface A Review. J. Liq. Chromatogr. R. T. 2006, 19, 2723-2752]. The functional elements would need to be modified to display these silane functional groups for covalent attachment to SiO₂. Polymer particles may also be formulated for surface modification, provided they display reactive functional groups that can be coupled to the functional elements. However, due to the low mass and volume ratio of functional rlements in this formulation, it is expected to be less effective for achieving high optical density in the photoactivated state due to low dye loading, compared to Formulations A and B, as well as Formulations D and E, presented below.

Formulation D. Core-shell Particle. Core-shell particles include formulations of core fluid/polymer shell, core fluid/inorganic shell, core polymer or gel/polymer shell, and core polymer or gel/inorganic shell. A convenient inorganic shell in this formulation is silica because it renders inorganic particles more biocompatible [Gerion, D.; Pinaud, F.; Williams, S. C.; Parak, W. J.; Zanchet, D.; Weiss, S; Alivisatos, A. P. Synthesis and properties of biocompatible water-soluble silica-coated CdSe/ZnS semiconductor quantum dots. J. Phys. Chem. B 2001, 105, 8861-8871]. The core or shell polymers may constitute the same polymers as discussed in Formulation A. above, with PMMA and PDMS being preferred for their transparency and biocompatibility. Core-shell particles are also known as nanocapsules or microcapsules, especially when they contain fluid cores, and they may be produced by a variety of emulsion-polymerization techniques [Jamekhorshid, A.; Sadrameli, S. M.; Farid, M. A review of microencapsulation methods of phase change materials (PCMs) as a thermal energy storage (TES) medium. Renew. Sust. Energy Rev. 2014, 31, 531-542] as well as by microfluidic reactor approaches [Wang, J.-T.; Wang, J.; Han, J.-J. Fabrication of Advanced Particles and Particle-Based Materials Assisted by Droplet-Based Microfluidics. Small 2011, 7, 1728-1754] or spray drying techniques [Gharsallaoui, A.; Roudaut, G.; Chambin, O.; Voilley, A.; Saurel, R. Applications of spray-drying in microencapsulation of food ingredients: An overview. Food Research International 2007, 40, 1107-1121].

Advantageous compositions of Formulation D would comprise a crystalline or amorphous molecular-aggregate core of the kind described in Formulation B with a thin PMMA or PDMS shell to provide a protective barrier. A second advantageous composition of Formulation D would comprise a biocompatible liquid or gel core containing a concentration of P-type photochromic dye(s) optimized for visibility in the photoactivated state, where the biocompatible liquid or gel matrix may comprise water, biocompatible oils such as vegetable oils, geraniol, etc., or cross-linked polyacrylate organogel or hydrogel networks which are commonly used for biomedical applications [Esposito, C. L.; Kirilov, P.; Roullin, V. G. Organogels, promising drug delivery systems: an update of state-of-the-art and recent applications. J. Contr. Release 2018, 271, 1-20].

Formulation E. Mesoporous Silica Nanoparticles. Mesoporous silica nanoparticles (MSNPs) are highly developed as nanocarriers for drug delivery applications [Slowing, I. I.; Vivero-Escoto, J. L.; Wu, C.-W.; Lin, V. S.-Y. Mesoporous silica nanoparticles as controlled release drug delivery and gene transfection carriers. Adv. Drug Deliv. Rev. 2008, 60, 1278-1288]. Their widespread use and biocompatibility in many settings make them, likewise, attractive carriers for photochromic compounds and other functional elements [Asefa, T.; Tao, Z. Biocompatibility of mesoporous silica nanoparticles. Chem. Res. Toxicol. 2012, 25, 2265-2284; Tarn, D. et al., Mesoporous silica nanoparticle nanocarriers: biofunctionality and biocompatibility. Acc. Chem. Res. 2013, 46, 792-801]. However, in contrast with drug delivery, where the contents of the particle are meant to be released, the functional elements must be permanently contained in the case of intradermal photochromic microparticles. Therefore, an advantageous method is to covalently attach the functional elements to the SiO₂ surface using alkoxysilanes and halosilanes [Voort, Der, P. V.; Vansant, E. F. Silylation of the Silica Surface A Review. J. Liq. Chromatogr. R. T. 2006, 19. 2723-2752]. However, it is also possible to contain the functional elements within the pores as long as the pore openings at the surface are sufficiently blocked to eliminate mass transport (cargo release). An advantage of MSNPs over silica nanoparticles (Formulation C) is that their much higher surface area (which can exceed 1000 square meters per gram) allows for a higher density of functional elements to be adsorbed to the surface of each particle (ultimately leading to more vibrant-appearing inks and tattoos). A process for preparing particles comprising photoactive dyes and silica or other ceramic particles is taught in U.S. Pat. No. 9,163,145 B2.

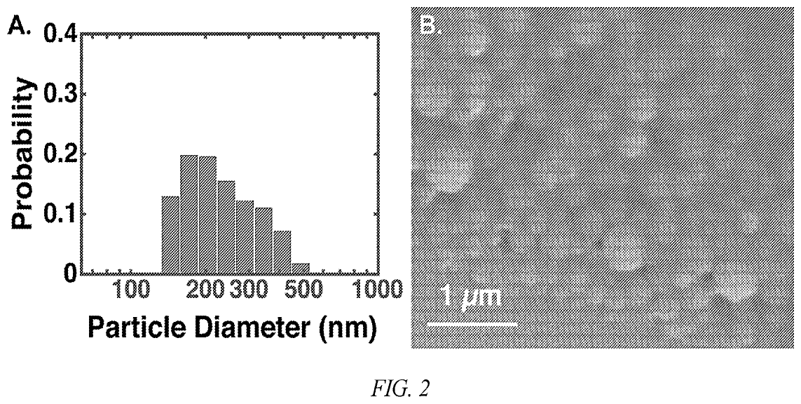

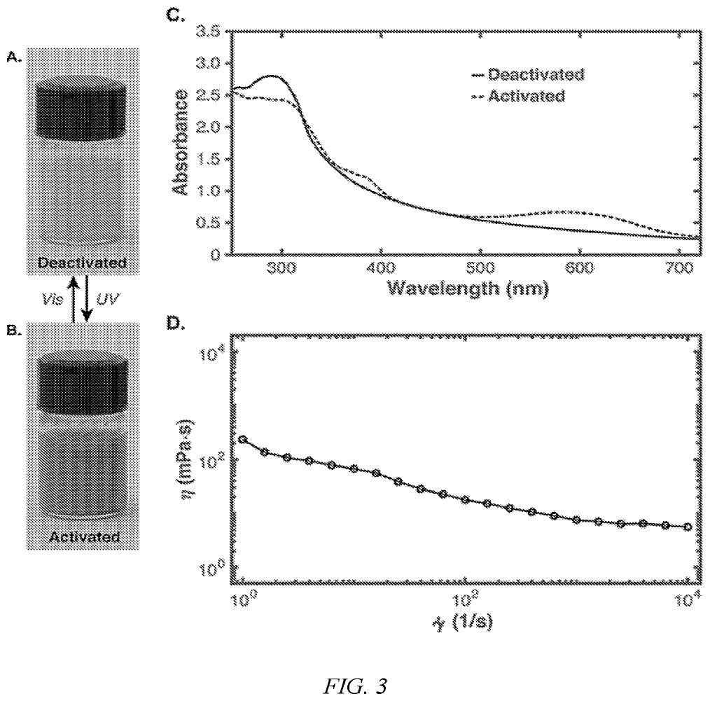

Example Procedure for the preparation of Bistable Photochromic PMMA microparticles. A solid powder of 1.2-Bis(2-methyl-5-phenyl-3-thienyl)-perfluoro-cyclopentenediarylethene (photochromic dye DAE-0001, Yamada Chemical) was mixed with PMMA at a mass ratio of 10:90, and this mixture was dissolved in dichloromethane at a concentration of 6% m/v. This solution was added dropwise to a solution of poly(vinyl alcohol) (PVA) in water (0.1 wt %) at RT to a concentration of 7.5% v/v. The resulting biphasic mixture was shaken briefly to form an emulsion, then horn sonicated for 15 minutes. The emulsion was transferred to a flask with a stir bar and stirred vigorously at room temperature. After 12 hours, the reaction was returned to room temperature and the particle suspension was transferred to a centrifugation tube. The particles were rinsed over several cycles of centrifugation, decanting the supernatant, and re-filling with purified water. The size distribution (FIG. 2A) of the particles was estimated using an Accusizer 780 optical particle sizer (NICOMP Particle Sizing Systems), and their absorption data (FIG. 3B) were collected using a Cary 5000 UV-Vis-NIR spectrophotometer (Agilent). The microparticles may be stored after synthesis as a wet or dry slurry.

Example Procedure for the preparation of Bistable Photochromic PDMS microparticles. Ultraviolet-absorptive microparticles of Formulation A, comprising P-Type photochromic dye dispersed in a PDMS matrix, was prepared. A PDMS pre-polymer resin was prepared using a two-part Sylgard 184 silicone elastomer kit (Dow Inc.) at a 10:1 base:catalyst mass ratio. An organic solution containing the diarylethene dye DAE-0001 (Yamada Chemical) such that the concentration of the dye was 1 mg/mL in the prepolymer. After 5 minutes of vigorous mixing. reverse-osmosis (RO) purified water was added to this pre-polymer / dye resin to obtain a biphasic mixture with a 4:1 water:resin mass ratio. TWEEN-80 (Sigma-Aldrich) surfactant was added to this biphasic mixture at a 1% mass ratio. The mixture was sonicated in an ultrasonic water bath (Branson M-1800) at room temperature for 5 minutes to generate an emulsion. A stir bar was added to the emulsion vessel and the solution was stirred at ˜1000 rpm at a temperature of 80 ° C. After 12 hours, the reaction was returned to room temperature and the particle suspension was transferred to a centrifugation tube. The particles were rinsed over several cycles of centrifugation, decanting the supernatant, and re-filling with purified water. The microparticles may be stored after synthesis as a wet or dry slurry.

### EXAMPLE 2—MULTISTABLE PHOTOCHROMIC MICROPARTICLE INKS

The bistable or multistable photochromic microparticles (see Example 1, above) may be dispersed in solvents or inks to prepare multistable photochromic inks. The ink formulations may be tailored for an intradermal delivery method, such as that described below, which can include a variety of tattooing/permanent makeup methods and microneedle or needle patches.

Tattoo and Permanent Make-Up Inks. In order to generate a liquid ink suitable for dermal implantation, the multistable photochromic microparticles are suspended in a fluid with or without additives. An exemplary fluid is water, although other biocompatible solvents such as alcohols (e.g., ethanol, isopropanol, glycerol, oligo-and polyethylene glycols) or oils (e.g., vegetable oils / triglycerides, geraniol, squalene, etc.) may also be employed. Appropriate additives for these inks include (i) antiseptics (e.g. alcohols) to prevent bacterial contamination, (ii) biocompatible surfactants (e.g., polysorbates) to stabilize the dispersions and adjust surface tension, (iii) thickening agents (e.g. xanthan gum, polyacrylates, polyglycols), [Petersen, H.; Roth, K. To Tattoo or Not to Tattoo? Chem. Unserer Zeit 2016, 50, 44-66] to increase viscosity and reduce pigment sedimentation rates (iv) thixotropic agents (e.g. silica) [Piccinini, P.; Pakalin, S.; Contor, L.; Bianchi, I.; Senaldi, C. Safety of tattoos and permanent make-up: Final report. European Commission Joint Research Centre Science for Policy Report 2016, 1-118] to promote shear thinning (v) preservatives/binding agents (e.g. polyethers, polyvinylpyrrolidinone) to help prevent the inks from drying and to help them bind to needles, (vi) astringents to minimize bleeding in the skin upon implantation, and/or (vii) anesthetics to minimize pain during ink implantation. The resulting inks can be sterilized with gamma radiation (preferred) or other means such as autoclave, heat, UV radiation, X-Ray radiation, or treatment with ethylene oxide prior to packaging and storage.

The bistable or multistable photochromic microparticles may be mixed with other bistable or multistable photochromic microparticles during ink formulation, either by pre-mixing dry or wet slurries of the different microparticles prior to ink formulation, or by mixing the individually formulated inks together to yield ink compositions containing two or more types of bistable or multistable P-type photochromic microparticles. These mixed-particle formulations can enable spectral tuning of the color response upon activation, as well as selective deactivation with specific wavelengths of light to access photo-multistable inks [Jin, Y. et al., Photo-Chromeleon: Re-Programmable Multi-Color Textures Using Photochromic Dyes. UIST '19 2019. 12 pp. New Orleans, LA, USA].

The bistable or multistable photochromic microparticles may also be mixed with T-Type photochromic microparticles suitable for tattoo inks, either by pre-mixing dry or wet slurries of the different microparticles prior to ink formulation, or by mixing the individually formulated inks together to yield ink compositions containing mixtures of P-type and T-type photochromic microparticles.

The bistable or multistable photochromic microparticles may also be mixed with standard pigments used in tattoo and permanent makeup inks. Examples of standard tattoo and permanent makeup pigment colors include white (e.g., titanium dioxide, zinc sulfide, barium sulhate), red (e.g., Pigment Red 22, 101, 122, 146, 170, 184, 188, 202, 210, 254), orange (e.g., Pigment Orange 13, 16, or 73), yellow (e.g., Pigment Yellow 14, 65, 74, 83, 97, or 194), blue (e.g., Pigment Blue 15 or 61), green (e.g., Pigment Green 7 or 36), and magenta/violet (e.g., Pigment Violet 1, 19, 23, 37). The combination of these standard pigments with a bistable or multistable photochromic microparticle tattoo inks will shift the color response of the ink toward the standard pigment's color in all of its photo-accessible states. The mixed multistable microparticle/pigment inks may be obtained by (i) dispersing multistable particles as wet or dry slurries directly into pre-formulated standard tattoo and permanent makeup inks, (ii) dispersing standard pigments as wet or dry slurries directly into pre-formulated bistable or multistable microparticle inks, or (iii) pre-mixing pigments and multistable microparticles as wet or dry slurries prior to ink formulation of these mixtures.

Example procedure for the preparation of bistable photochromic microparticle inks. A tattoo ink of a bistable photochromic PDMS microparticle of Formulation A (see Example 1, above) was created by suspending the wet slurry in reverse osmosis purified water at a mass ratio of 30% in the presence of PVA (0.1% w/v). The suspension was vigorously shaken by hand in a scintillation vial for 30 seconds. The ink was characterized by characterized by photography (FIG. 3A-B), UV-Vis spectroscopy (FIG. 3C), shear rheology (FIG. 3D). The ink remained well-dispersed on the timescale of hours. Although not employed in this example, advantageous formulations include glycerol or polyethylene glycol (molecular weight 1000, Sigma-Aldrich) added at a ratio of 1%-30% as an antiseptic agents, thickeners, and binders to improve the stability and transferablility of the bistable photochromic nanoparticle ink.

Microneedle Tattoo Inks. An emerging technology that should prove suitable for delivering materials, such as the bistable or multistable photochromic microparticle ink, into the dermis is the microneedle patch, a type of device with many possible configurations of micro-structured protrusions that penetrate the epidermis, which is typically targeted for transdermal drug delivery and vaccine applications [Prausnitz, M. R. Engineering Microneedle Patches for Vaccination and Drug Delivery to Skin. Annual Rev. Chem. Biomol. Eng. 2017, 8, 177-200]. U.S. Pat. No. 6,565,532 BI teaches a microneedle apparatus used for marking skin and for dispensing semi-permanent subcutaneous makeup. While these devices have not appeared on the market, it may be possible to use them for intradermal implantation of photochromic microparticles. The ink formulations for these microneedle patches will consist of a suspension of photochromic microparticles (optionally mixed with pigments) in a fluid containing polymer, pre-polymer, or molecular precursors to the matrix of the microneedle delivery method. For example, an advantageous formulation would employ dissolving microneedle arrays [see e.g., Bediz, B. et al., Dissolvable Microneedle Arrays for Intradermal Delivery of Biologics: Fabrication and Application. Pharm. Res. 2013, 31, 117-135], since this formulation of microneedle patches is optimized for delivering relatively high amounts of material compared to other microneedle patch formulations. The carrier matrix for dissolving microneedle arrays is advantageously a non-toxic material of sufficient strength to penetrate the epidermis, but sufficiently water soluble to dissolve rapidly in the interstitial fluid of the dermis and thus release its contents. Examples of suitable carriers for microneedle invisible ultraviolet-absorptive microparticle inks include polyvinylpyrrolidinone or polyvinyl alcohol and their liquid pre-polymers, or aqueous solutions of carboxymethyl cellulose, trehalose, maltodextrin, galactose, glucose, hyaluronic acid, and silk, which solidify within microneedle molds upon curing or drying, respectively.

Microneedles, having needle width and depth dimensions <1 mm, may be too small for implanting materials permanently in the dermis, since it has a mean thickness of ˜2 mm and can reach thicknesses up to 4 mm [Oltulu, P.; Ince, B.; Kokbudak, N.; Findik, S.; Kilinc, F. Measurement of epidermis, dermis, and total skin thicknesses from six different body regions with a new ethical histometric technique. Turk. J. Plast. Surg. 2018, 26, 56-61], and tattoo machines penetrate up to 4 mm into the skin [Petersen, H.; Roth, K. To Tattoo or Not to Tattoo? Chem. Unserer Zeit 2016, 50, 44-66]. Dissolving needles of larger dimensions (>1 mm) could be prepared by similar methods, using masters and molds with larger-scale features, and may be more suitable for use in applications as proposed in the present invention.

### EXAMPLE 3-IMPLANTATION METHODS FOR MULTISTABLE PHOTOCHROMIC MICROPARTICLE TATTOOS

A safe and effective amount of multistable photochromic microparticle tattoos may be implanted by a variety of methods, typically involving a needle or array of needles, dipped in multistable photochromic microparticle dispersions (see example 2, above). The ink-coated needles can repeatedly puncture the skin in order to break through the epidermal barrier and deliver the ink material into the dermis. Inserting the needle or needles into the skin may be performed by hand according to a number of ancient indigenous tattooing traditions, including tapping (tatau, Polynesia), raking (tebori, Japan), threading/stitching with needle and thread (North America), and laceration followed by ink rubbing (Europe). [Krutak, L.; Deter-Wolf, A. (Eds.). Ancient Ink: The Archaeology of Tattooing 2017. Seattle; London: University of Washington Press] An advantageous method is to attach a needle array to a modern motorized tattoo or permanent make-up machine, which improves efficiency and minimizes pain compared to hand-driven methods. Needle-free tattoo machines that inject tattoo ink droplets into the skin at sufficiently high velocity to penetrate into the dermis have been described in academic literature [Oyarte Gálvez, L.; Brió Pérez, M.; Fernández Rivas, D. High speed imaging of solid needle and liquid micro-jet injections. J. Appl. Phys. 2019, 125, 144504-13; Cu, K.; Bansal, R.; Mitragotri, S.; Rivas, D. F. Delivery Strategies for Skin: Comparison of Nanoliter Jets, Needles and Topical Solutions. Ann. Biomed. Eng. 2019, 2028-2039], and are taught in U.S. Pat. No. 6,689,095 B1 by Garitano and Garitano, L. To the extent compatible with standard tattoo inks, these machines may also be employed in the present application.

Alternatively, the ink may be formulated into a dissolving microneedle or needle patch in a PDMS mold as described by [Bediz, B. et al., Dissolvable Microneedle Arrays for Intradermal Delivery of Biologics: Fabrication and Application. Pharm. Res. 2013, 31, 117-135]. A patch can be employed that is inserted in the skin only once and held in place for sufficient time to allow the multistable photochromic microparticle ink to be released in the interstitial fluid of the dermis.

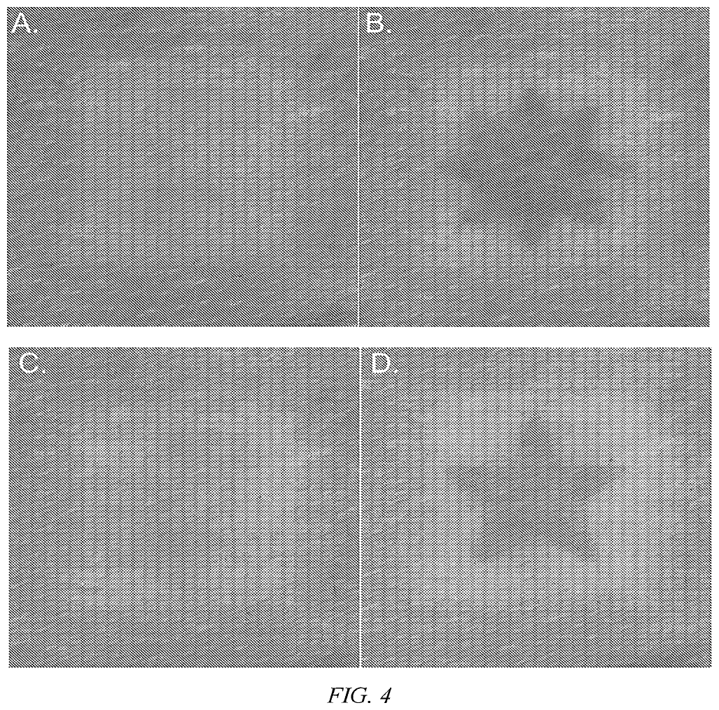

Example procedure for implantation of multistable photochromic microparticle inks. Using an ex vivo porcine skin model, a multistable photochromic microparticle tattoo was implanted with a rotary tattoo machine (Dragonhawk) equipped with a steel 9RS tattoo needle array, dipped in a tattoo ink comprising an aqueous dispersion of approximately 10 wt % PDMS-based bistable photochromic microparticles (described in the examples 1 and 2, above) at a drive power of 7 V over an area of 1 square centimeter until a tattoo of uniform appearance was obtained. The skin sample was cleaned with isopropanol before and after tattooing. Photographs of this photochromic particle tattoo undergoing cycles of writing and erasing with UV and red light, respectively, are shown in FIG. 4, verifying that the tattoos function as photochemically bistable tattoos.

### EXAMPLE 4—APPLICATIONS OF ULTRAVIOLET-ABSORPTIVE MICROPARTICLE TATTOOS

### Uses and Benefits of the Innovation

Multistable photochromic microparticle tattoos may be used in a new form of semi-permanent or permanent body art which can be frequently re-programmed using different wavelengths of light, photo-switchable anatomical markers, and, in certain formulations, short-term colorimetric UV detectors and dosimeters.

### Multistable Photochromic Tattoos for Photochemically Rewritable Body Art

Pigment tattoo and permanent makeup inks are utilized by hundreds of millions of people around the world, including approximately one-quarter of the U.S. adult population for the administration of permanent body art and cosmetics [Piccinini, P.; Pakalin, S.; Contor, L.; Bianchi, I.; Senaldi, C. Safety of tattoos and permanent make-up: Final report. European Commission Joint Research Centre Science for Policy Report 2016, 1-118]. Bistable or multistable photochromic tattoo and permanent makeup inks, which can be implanted in the same manner as conventional tattoos and permanent makeup, can be used instead of, in combination with, or in addition to these widely used inks to generate body art or permanent cosmetic markings that change color in response to different types of illumination. For example, the images in FIG. 4 indicate how a tattoo design can be reversibly programmed, erased, and re-programmed with UV light, red light, and UV light, respectively, allowing the user to change their tattoo design using only irradiation with light, rather than more invasive and irreversible procedures such as tattooing (known in this context as “cover-ups”) or laser ablation.

The body art and permanent makeup tattoos made with bistable or multistable photochromic microparticle inks may be programmed into specific designs or patterns using (i) monochromatic or polychromatic light sources in combination with transparency masks that cover portions of the tattooed area during exposure (as in FIG. 4), (ii) projectors that cast patterns or images of differentiated wavelengths of light onto the skin such as the protocol described by Jin et al. [Jin, Y.; Qamar, I.; Wessely, M.; Adhikari, A.; Bulovic, K.; Punpongsanon, P.; Mueller, S. Photo-Chromeleon: Re-Programmable Multi-Color Textures Using Photochromic Dyes. UIST '19 2019. 12 pp. New Orleans, LA, USA]), or (iii) lasers that allow small regions of the tattoo to be locally activated or deactivated, enabling the implementation of a raster or pixel-by-pixel approach to programming the tattoo design with an appropriate computer numerical control system, or “hand-drawn” designs when the lasers are manipulated manually.

The body art and permanent makeup tattoos made with bistable or multistable photochromic microparticle inks containing mixtures of P-type and T-type photochromic dyes may be programmed to undergo more dynamic color changes that evolve in time as the lighting changes. For example, a T-type photochromic ink that appears yellow when activated by UV radiation, and rapidly thermally deactivates back to a colorless state upon removal of UV irradiation, in combination with a P-type photochromic ink that is colorless in the ground state and cyan in the UV-activated photostationary state, would appear either (i) colorless (colorless +colorless) prior to UV irradiation, (ii) green (yellow+cyan) under active UV irradiation, and (iii) cyan (colorless +cyan) upon removal of UV, but prior to deactivation of the cyan ink via red light. A multitude of combinations is possible according to these principles, and these can further be combined with conventional tattoo inks and the methods described above to yield more color combinations and complexity of design.

### Photochemically Activated Anatomical Markers for Medical Applications

Dermatologists routinely use intradermal pigments to demarcate biopsy sites that test positive for cancer or other disease that may require complete removal at a later date by a surgeon [Goldman, L.; Richfield, D.; Kubitz, D. Small Biopsy With Tattoo Identification of Tissue. Archives of Dermatology 1964, 90, 195-196; [Jalgaonkar, A. et al., Preoperative biopsy tract identification using india ink skin tattoo in tumous surgery. Orthopaedic Proceedings 2012, 94-B:SUPP_XXXVII, 321; Chuang, G. S.; Gilchrest, B. A. Ultraviolet Fluorescent Tattoo Location of Cutaneous Biopsy Site. Dermatol. Surg. 2012, 38, 479; Choi, J. et al., Cross-Linked Fluorescent Supramolecular Nanoparticles as Finite Tattoo Pigments with Controllable Intradermal Retention Times. ACS Nano 2017, 11, 153-162]. Since the patient's surgery may occur months after the biopsy, these intradermal markings are intended to reduce the uncertainty and error associated with the surgeon's correct identification of the surgical site(s). Especially on areas of skin that are highly visible in public, medical practitioners may use “invisible” intradermal pigments that fluoresce in appropriate lighting such as ultraviolet or “black” light. These fluorescent pigments minimize the visibility of the biopsy site marker in the patient's skin in normal indoor and outdoor lighting conditions. However, the composition and safety of these pigments are often unknown, and the fluorescence may be difficult to detect by the naked eye in well-lit environments. The bistable or multistable photochromic particles, inks, and tattoos described in Examples 1-3, above, may provide a convenient and potentially safer alternative to these biopsy marking procedures.

Intradermal pigments are also employed routinely in radiological oncology to aid in beam alignment at the anatomical site. For the same reasons in the case of dermatologic surgery described above, the bistable or multistable photochromic pigments described in this invention may be employed to change the visibility of these anatomical markers.

In particular, particle and ink formulations containing only P-type photochromic dyes that are colorless in the ground state and visible in a UV-activated state (as demonstrated with photochromic dye 1,2-bis(2-methyl-5-phenyl-3-thienyl)-perfluorocyclopentene in the above examples and FIGS. 1-4) are advantageous for applications as anatomical biopsy site markers, since the corresponding photo-bistable biopsy markers may remain colorless until activated by a UV lamp. Thus, when appropriate bistable or multistable photochromic inks are administered according to any of the procedures described in Example 3, above, these photochromic intradermal particles may have low visibility in the patient's skin in indoor lighting, and become visible in order to facilitate biopsy site identification by medical practitioners only after a brief period of exposure to a UV lamp or sunlight.

### Intradermal UV Dosimetry.

Ultraviolet (UV) radiation is the main risk factor for skin cancer (both melanoma and non-melanoma), which is the most common malignancy (more common than all other cancers combined) in the United States and other predominantly light-skinned populations worldwide [Diepgen, T. L.; Mahler, V. The epidemiology of skin cancer. Br. J. Derm. 2002, 146, 1-6; Rogers, H. W.; Weinstock, M. A.; Feldman, S. R.; Coldiron, B. M. Incidence Estimate of Nonmelanoma Skin Cancer (Keratinocyte Carcinomas) in the US Population, 2012. JAMA Dermatol. 2015, 151, 1081-1086]. Most of the UV rays transmitting through the earth's atmosphere are UVA (320-400 nm wavelength), while a small amount of UVB rays (280-320 nm wavelength) also reach the earth's surface. Exposure to both UVA and UVB leads to cumulative skin damage over time, increasing skin cancer risk and aging rates [Taylor, C. R.; Stern, R. S.; Leyden, J. J.; Gilchrest, B. A. Photoaging/Photodamage and Photoprotection. J. Am. Acad. Dermatol. 1990, 22, 1-15; Koh, H. K.; Geller, A. C.; Miller, D. R.; Grossbart, T. A .; Lew, R. A. Prevention and Early Detection Strategies for Melanoma and Skin Cancer: Current Status. Arch. Dermatol. 1996, 132, 436-443]. These resources verify that UVB radiation is the primary cause of sunburn and the main risk factor for melanoma (one of the least common but most lethal skin cancers) and other skin cancers, while the more deeply penetrating UVA rays are associated with skin aging and further increase the risk of the most common keratinocyte carcinomas.

Personal UV dosimetry facilitates protective interventions against skin cancer and other UV-associated diseases by providing quantitative data about cumulative UV exposure at the site of the detector of a wearable UV dosimeter. [Foller, P.; Fritz, I.; Olguin, C.; Wrobel, S.; Le Maitre, C.; Kang. E. R.; Tibbits, S. J. E. Sensing of solar ultraviolet radiation by wearable colorimetry. U.S. Patent US20200149960A1. Filed 18 Jun. 2018; Davis, A.; Deane, G. H. W.; Diffey, B. L. Possible dosimeter for ultraviolet radiation. Nature 1976, 261, 169-170]. Wearable UV dosimeters can be based on a variety of materials, including polysulfone, UV-reactive dyes embedded in polymer films, and zinc oxide nanowires [Zou, W.; Sastry, M.; Gooding, J. J.; Ramanathan, R.; Bansal, V. Recent Advances and a Roadmap to Wearable UV Sensor Technologies. Adv. Mater. Technol. 2020, 5, 1901036]. However, wearable dosimeters suffer from some limitations: electronic dosimeters require batteries that may expire while also being relatively bulky and expensive, while thin-film wearable dosimeters have limited shelf life and single-use designs that lead to accumulated wastes and costs over time. Furthermore, in wearable dosimeters, UV sensing occurs on the surface of the skin, and may therefore overestimate UV dosage beneath the skin's surface where most UV-vulnerable tissues are located. Intradermal UV dosimeters may overcome these limitations. Ideally, the information recorded by an intradermal UV dosimeter may be read colorimetrically and then reset and re-used, in order to avoid repeated dermal implantation of new dosimetry materials; however, the existing wearable UV dosimeter materials do not meet this requirement.

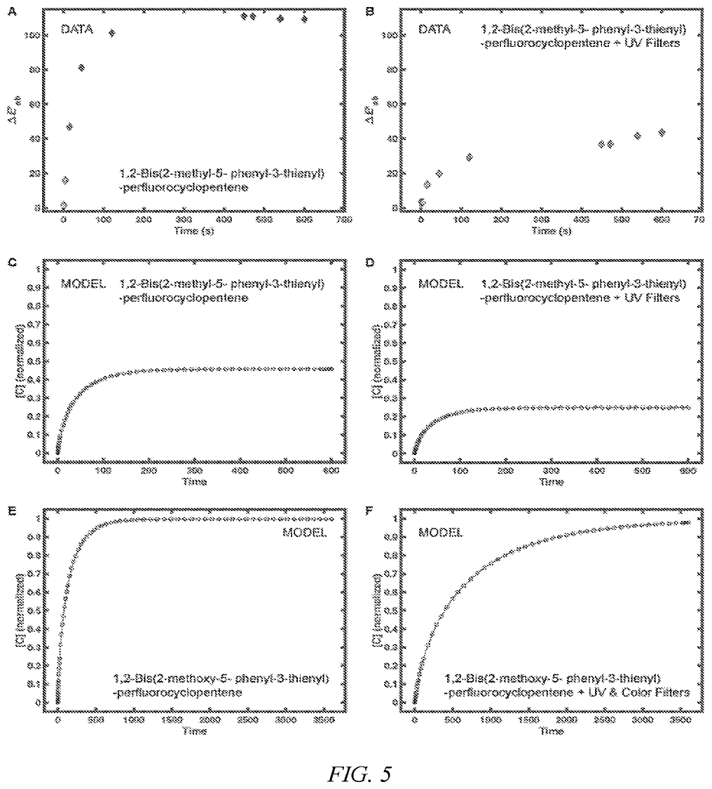

To enable repeatable UV dosimetry with intradermal P-type photochromic microparticles, an advantageous formulation employs a colorless P-Type photochromic dye that, when activated by UV light, forms a colored photostationary state that can be deactivated only by visible or near-infrared light. In this case, the efficiency of UV activation must vastly exceed that of visible or near-IR deactivation (cycloreversion in the case of diarylethenes), since the UV light in sunlight is accompanied by large amounts of broad-spectrum visible and near-IR light that may deactivate the photochromic dye before its color can be measured for dosimetry purposes. Conveniently, if the photochemical rate constant, quantum yield, and wavelength-dependent molar absorptivities of the dye are known, then the dye's coloration may be predicted as a function of the spectral distribution, intensity, and illumination time of the light source, as demonstrated in FIG. 5. For example, in a dye that is activated to a colored state by UV light and deactivated to a colorless state by visible light, we model the relative concentration of the colored state (photoactivated or photostationary state) as a function of time using an AM1.5G standard to simulate the solar spectral distribution of sunlight in the earth's atmosphere near sea level, using the following Equation:

$$\frac{d [ C ]}{dt} = {\sum }_{λ = 280}^{700} ( {ϕ}_{c} \frac{{ε}_{s , λ} ( [ S ] )}{{ε}_{s , λ} ( [ S ] ) + {ε}_{c , λ} ( [ C ] )} - {ϕ}_{s} \frac{{ε}_{c , λ} ( [ C ] )}{{ε}_{s , λ} ( [ S ] ) + {ε}_{c , λ} ( [ C ] )} ) * \text{} {I}_{λ} ( \text{} 1 - {10}^{- ( {ε}_{s , λ} ( [ S ] ) + {ε}_{c , λ} ( [ C ] ) * l )}$$

where [C] is the concentration of the photoactivated species (photostationary state), [S] is the concentration of the ground-state species, ε_{S,λ} is the molar absorptivity of the ground-state species at wavelength λ, ε_{C,λ} is the molar absorptivity of the photoactivated species at wavelength λ, φs is the quantum yield of the cycloreversion or deactivation, φ_{C} is the quantum yield of photoactivation, I_{λ} is the irradiance at wavelength λ, and l is the path length. In this model, I_{λ} is based on empirical solar irradiance data in the ASTM G173-03 standard solar spectrum. The graphs in FIG. 5C and FIG. 5D apply this equation based on empirical absorptivity and quantum yield data for P-type photochromic dye DAE-0001 used in Examples 1-3 above. The simulations agree well with measured data of its UV activation in natural sunlight in Boulder, Colorado, as shown in in FIG. 5A and FIG. 5B.

Varying the constants that define the molar absorptivity, rate, and quantum yield for the activation and deactivation reactions, it is found that the kinetics of activation become sufficiently slow for practical UV dosimetry in sunlight (minutes to hours, as shown in FIG. 5F) when the quantum yield of the deactivation (cycloreversion) reaction driven by visible light is reduced to <10⁻⁴ and the UV and visible light irradiance are reduced by factors of 0.66 and 0.5, respectively. In practice, these low deactivation quantum yields can be achieved by appropriate selection of the photochromic dye(s), while the reduced UV and visible irradiance can be achieved by mixing the bistable photochromic dye or particle with UV and color filters in the form of dyes and pigments, as described in Example 1 above.

A suitable P-type photochromic dye for UV dosimetry may be 1,2-bis(2-methoxy-5-phenyl-3-thienyl)-perfluorocyclopentene (DAE-C1, Yamada Chemical), since its UV-activated cyclization has a quantum yield of 0.44, while its visible-activated cycloreversion has a quantum yield of <0.00002, and the absorptivities of both ring-open and ring-closed states are of the same order of magnitude [Shibata, K.; Kobatake, S.; Irie, M. Extraordinarily low cycloreversion quantum yields of photochromic diarylethenes with methoxy substituents. Chem. Lett. 2001, 30, 618-619]. The simulations graphed in FIG. 5E and FIG. 5F are based on empirical absorptivity and quantum yield data for this compound. Another suitable P-type photochromic dye for UV dosimetry may be 1,2-bis[2-methyl-5-(4-phenylbuta-1,3-dienyl)thien-3-yl]-perfluorocyclopentene, since its UV-activated cyclization has a quantum yield of 0.6, while its visible-activated cycloreversion has a quantum yield of 0.00003, and the absorptivities of both ring-open and ring-closed states are of the same order of magnitude [Bens, A. T.; Frewert, D.: Kodatis, K.; Kryschi, C.; Martin, H.-D.; Trommsdorff, H. P. Coupling of Chromophores: Carotenoids and Photoactive Diarylethenes—Photoreactivity versus Radiationless Deactivation. Eur. J. Org. Chem. 1998, 2333-2338]. Many other P-type dyes with similar quantum yield ratios of activation and deactivation are suitable candidates for UV dosimetry by the method(s) described herein.

The tattoo color may be quantified in order to perform quantitative UV dosimetry using the intradermal P-type photochromic dyes with low deactivation/cycloreversion quantum yields. In order to quantify the tattoo color, the tattoo may be photographed with a camera, including those found in webcams and mobile cellular devices, and subjected to an image processing procedure. The image processing procedure may be performed manually or automated by a software application. In a manual procedure, the area of the tattoo may be characterized before UV activation, and after complete UV activation, in a particular digital color space. Examples of suitable color spaces include RGB, CMYK, HSV, CIE1931, and CIELAB systems. In FIG. 5A and FIG. 5B, empirical data for color change is plotted using CIELAB chromaticity space, as quantified by the metric ΔE*_{ab}. The two photographs of the fully deactivated and fully activated tattoo define the full range of sensitivity for the dosimeter tattoo, which can then be used to calibrate the photographs against a modeled or empirically measured “standard” sensitivity curve as depicted in FIG. 5. Once this calibration is complete, the UV dose that has been introduced to the intradermal tattoo can be quantified whenever the color-shift (e.g. ΔE*_{ab} value) of the tattoo is less than its maximum by mapping its observed value to the calibrated the standard curve, where the UV dose is known as a function of color. The simulated curve in FIG. 5F indicates that an appropriately designed system can reach 85% activation in 1092 seconds (18.2 minutes), which is longer than the time it takes to receive a standard erythemal dose of solar radiation in unprotected skin in direct sunlight on the earth's surface at ASTM G03-173 standard irradiance levels. To facilitate intradermal UV dosimetry, software tools may be developed to automate the manual calibration and colorimetry process described here using mathematical models or machine learning/artificial intelligence platforms.

The intradermal UV dosimetry procedure described above may also be accomplished qualitatively by naked-eye comparison of the tattoo with color charts that correlate color with UV dose, as well as by video analysis (as opposed to photographic analysis) of tattoo color, accounting for the rate of color change when a known UV or visible light dose is applied via a source with a known output spectrum.

As our model demonstrates, the density and wavelength sensitivity of the UV and color filters can be used to tune the activation kinetics of these intradermal UV dosimeters to meet the needs of the user. For example, a tattoo that reaches full activation after one standard erythemal dose may be useful for managing UV exposure for vitamin D production without exceeding the limits beyond which skin cancer risk begins to increase. The dosimeter tattoo can then be “reset” with red light (in the case of DAE-C1) or another wavelength of light that promotes deactivation on an hourly or daily basis to repeat the dosimetry monitoring process as needed.

### DEFINITIONS

The term “administration” and variants thereof (e.g., “administering” a compound) in reference to a compound of the invention means introducing the compound into the system of the subject in need of treatment, such as via injection into the dermal layer of the skin of the subject. When a compound of the invention is provided in combination with one or more other active agents, “administration” and its variants are each understood to include concurrent and sequential introduction of the compound and other agents.

As used herein, the term “composition” is intended to encompass a product comprising the specified ingredients in the specified amounts, as well as any product which results, directly or indirectly, from combination of the specified ingredients in the specified amounts.

A “pharmaceutically acceptable” component is one that is suitable for use with humans and/or animals without undue adverse side effects (such as toxicity, irritation, and allergic response) commensurate with a reasonable benefit/risk ratio.

A “safe and effective amount” refers to the quantity of a component that is sufficient to yield a desired therapeutic response without undue adverse side effects (such as toxicity, irritation, or allergic response) commensurate with a reasonable benefit/risk ratio when used in the manner of this invention.

As used throughout the entire application, the terms “a” and “an” are used in the sense that they mean “at least one”, “at least a first”, “one or more” or “a plurality” of the referenced components or steps, unless the context clearly dictates otherwise. For example, the term “a cell” includes a plurality of cells, including mixtures thereof.

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range.

As used herein, the term “comprising” is intended to mean that the products, compositions and methods include the referenced components or steps, but not excluding others. “Consisting essentially of” when used to define products, compositions and methods, shall mean excluding other components or steps of any essential significance. Thus, a composition consisting essentially of the recited components would not exclude trace contaminants and pharmaceutically acceptable carriers. “Consisting of” shall mean excluding more than trace elements of other components or steps.

As used herein, the term “bistable” refers to a color that has two stable color states. As used herein, the term “multistable” refers to a color that has two or more stable color states.

As used herein, the term “photochromic” refers a substance or composition that is capable of changing color on exposure to radiant energy (such as light). Photochromism is the reversible transformation of a chemical species between two forms by the absorption of electromagnetic radiation, where the two forms have different absorption spectra. In simple terms, this can be described as a reversible change of colour upon exposure to light.

As used herein, a “P-type photochromic dye” is a compound that meets the following two criteria: (i) the compound undergoes a photochemical reaction that changes its spectral absorbance profile when activated by a particular wavelength or range of wavelengths of light, and (ii) the photochemical reaction undergone by the compound is not thermally reversible but can be reversed by photochemical de-activation at a different wavelength or range of wavelengths than those used for activation.

By “suitable for injection” it is meant that the particles are pharmaceutically acceptable and exhibit little to no toxicity, immunogenicity, or teratogenicity.

Kits for practicing the methods of the invention are further provided. By “kit” is intended any manufacture (e.g., a package or a container) comprising at least one reagent, e.g., a pH buffer of the invention. The kit may be promoted, distributed, or sold as a unit for performing the methods of the present invention. Additionally, the kits may contain a package insert describing the kit and methods for its use. Any or all of the kit reagents may be provided within containers that protect them from the external environment, such as in sealed containers or pouches. The advantages set forth above, and those made apparent from the foregoing description, are efficiently attained. Since certain changes may be made in the above construction without departing from the scope of the invention, it is intended that all matters contained in the foregoing description or shown in the accompanying drawings shall be interpreted as illustrative and not in a limiting sense.

All references cited in the present application are incorporated in their entirety herein by reference to the extent not inconsistent herewith.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall there between. Now that the invention has been described.

1.-51. (canceled)

52. A bistable or multistable photochromic particle suitable for intradermal use comprising a biocompatible polymer in combination with at least one P-type photochromic dye.

53. The bistable or multistable photochromic particle of claim 52, wherein the P-type photochromic dye is diarylethene, 1,2-Bis(perfluorocyclopentene)-perfluorocyclopentene, 1,2-Bis[2-methyl-5-(4-phenylbuta-1,3-dienyl)thien-3-yl]-perfluorocyclopentene, fulgide, fulgimide, naphthopyran, hydrazone, or combinations thereof.

54. The bistable or multistable photochromic particle of claim 52, including a stable UV- or Visible-absorptive material to tune optical spectral sensitivity and color appearance.

55. The bistable or multistable photochromic particle of claim 54, wherein the UV-absorptive material is hydroxybenzophenone, hydroxyphenyl-s-triazine, 2-(2-hydroxyphenyl)benzotriazole, oxalanilide, Aminobenzoic acid, Avobenzone, Cinoxate, Dioxybenzone, Homosalate, Meradimate, Octocrylene, Octinoxate, Octisalate, Oxybenzone, Padimate O, Ensulizole, Sulisobenzone, Cerium Dioxide, Titanium dioxide, Trolamine salicylate, Zinc oxide, or combinations thereof, including layered double hydroxides derived thereof.

56. The bistable or multistable photochromic particle of claim 54, wherein the visible-absorptive material is an azo dye, perylene, anthraquinone, cyanine, triarylmethine, or commercial pigments including the groups of Pigment Red, Pigment Orange, Pigment Yellow, Pigment Blue, Pigment Green, Pigment Violet, Pigment Black, or Pigment White, or combinations thereof.

57. The bistable or multistable photochromic particle according to claim 52, including a photo-stabilizer to inhibit photodegradation of the photochromic dye and polymer.

58. The bistable or multistable photochromic particle of claim 57, wherein the photo-stabilizer is a hindered amine comprising 2,2,6,6-tetramethylpiperidine, an alkylated or hydroxylamine analog of 2.2,6,6-tetramethylpiperidine, a polymer containing 2.2,6,6-tetramethylpiperidine moieties, or combinations thereof.

59. A bistable or multistable photochromic particle of claim 52, where the particle comprises a polymer particle, a molecular aggregate, an inorganic nanoparticle, an inorganic microparticle, a surface coated nanoparticle, a surface coated microparticle, a core-shell nanoparticle, a core-shell microparticle, a mesoporous nanoparticle, or a mesoporous microparticle.

60. The bistable or multistable photochromic particle of claim 52, wherein the biocompatible polymer is poly(methylmethacrylate) (PMMA), polylactic acid (PLA), poly(lactic-co-glycolic acid) (PLGA), poly(dimethylsiloxane) (PDMS), polyethylene glycol (PEG), Melamine-formaldehyde, Methacrylamide chitosan, or combinations thereof.

61. The bistable or multistable photochromic particle of claim 52, wherein the particle is suspended in a biocompatible solvent comprising water, alcohols, oils, or combinations thereof.

62. The bistable or mulstistable photochromic particle of claim 52, including an additive comprising an antiseptic, a biocompatible surfactant, a thickening agent, a thixotropic agent, a preservative, a binding agent, an astringent, an anesthetic, or combinations thereof.

63. The bistable or multistable photochromic particle of claim 52, including polyvinyl alcohol surfactant at ratio of <1.0% (v/v) to stabilize the particle, and polyethylene glycol (molecular weight 1000), 1,2-propanediol, 1,3-propanediol, glycerol, ethanol, or isopropanol added at a ratio of 1%-30%, whereby the alcohol additive comprises an antiseptic agent, thickener, binder, or combinations thereof.

64. The bistable or multistable photochromic particle of claim 52, wherein the particle is combined with an ink or pigment.

65. The bistable or multistable photochromic particle of claim 52, including one or more T-type photochromic dyes comprising spiropyran, spirooxazine, or combinations thereof.

66. A method of rendering the skin photochromically bistable or multistable, comprising:
(a) providing a composition including a bistable or multistable photochromic particle suitable for intradermal use, the particle comprising a biocompatible polymer in combination with a P-type photochromic dye; and
(b) implanting the composition intradermally.

67. The method of claim 66, wherein the bistable or multistable photochromic particle is intradermally implanted using a microneedle device.

68. The method of claim 67, wherein the microneedle device is a dissolving microneedle array comprising the photochromic particle suspended in polyvinylpyrrolidinone, polyvinyl alcohol, a liquid pre-polymer of polyvinylpyrrolidinone, a liquid pre-polymer of polyvinyl alcohol, an aqueous solution of carboxymethyl cellulose, an aqueous solution of trehalose, an aqueous solution of maltodextrin, an aqueous solution of galactose, an aqueous solution of glucose, an aqueous solution of silk, or combinations thereof.

69. The method of claim 66, wherein the composition is intradermally implanted using a liquid micro-jet injection system.

70. The method of claim 66, wherein the composition is intradermally implanted using an electric tattoo machine.

71. The method of claim 66, wherein the composition includes an ink or pigment.
